(19) United States
(12) Patent Application Publication
(10) Pub. No.: US 2016/0120793 A1
(43) Pub. Date: May 5, 2016

(54) Oral Healthcare Product

(71) Applicant: ALEQUIDENT LIMITED, Auckland (NZ)

(72) Inventors: Hisham Abdalla, Kohimarama (NZ); Andrew Lavrent, Greenlane (NZ)

(73) Assignee: ALEQUIDENT LIMITED, Auckland (NZ)

(21) Appl. No.: 14/891,563

(22) PCT Filed: May 19, 2014
(86) PCT No.: PCT/NZ2014/000089

§ 371 Date: Nov. 16, 2015

Related U.S. Application Data
(60) Provisional application No. 61/824,449, filed on May 17, 2013.

(51) Int. Cl. A61K 8/97; A61K 8/24; A61K 8/19; A61Q 11/00; A61K 8/34

(52) U.S. Cl. CPC A61K 8/97; A61Q 11/00; A61K 8/345; A61K 8/19; A61K 8/24; A61K 2800/5922

(57) ABSTRACT

The invention provides an oral healthcare product that includes a saliva activating agent, a pH adjusting agent, a source of bioavailable calcium ions, a source of bioavailable phosphate ions, and an antibacterial natural extract of totara. The invention also provides methods of using and preparing such compositions.

### FIELD OF INVENTION

The invention relates to products to maintain oral health. More particularly, though not solely, the invention relates to products to obtain and maintain beneficial environment in the oral cavity.

### BACKGROUND

Tooth decay and gum disease are prevalent worldwide. The financial, health, psychological and social impacts of these issues are of concern to individuals, governments and international organisations such as W.H.O. (World Health organisation).

Even in relatively wealthy countries where dental and oral care products are readily available there is still a problem with tooth decay and gum disease due to individuals either not complying with instructions associated with present dental and oral care products, or that the products are not used at the most effective time (for example, when or soon after a damaging practice or event takes place) or that the present products are not sufficiently effective or beneficial.

Dental and oral care products currently on the market that claim to treat or prevent tooth decay and gum disease often contain ingredients which can have negative side effects. For example many products contain propylene glycol which is a potential oral mucosa irritant. Many products also contain alcohols and sodium lauryl sulphate which are desiccating agents and known irritants. Ingredients which are desiccating dry out the mouth and reduce the naturally beneficial effect of saliva, especially in individuals with impaired saliva production or poor saliva quality (for example xerostomics, elderly, heavily medicated individuals, smokers, those with diets high in sugar, high in caffeine, high in alcohol and/or high in acidic drinks). Some oral care products can make the naturally neutral pH environment in the mouth acidic, which can cause demineralisation and erosion. In addition, some ingredients which are included as a means to thoroughly clean the teeth, (for example silica) can be too abrasive, resulting in the wearing away of the protective enamel on the teeth with prolonged use. Many dental and oral care products presently on the market state they are not to be swallowed, due to known potential toxicity.

It is an object of the present invention to provide improved oral health care products and methods, or to overcome the above shortcomings or address the above desiderata, or to at least provide the public with a useful choice.

### SUMMARY OF THE INVENTION

According to a first broad aspect of the invention, there is provided an oral healthcare product, comprising:
about 30% to about 98% w/w of a saliva activating agent,
a pH adjusting agent,
a source of bioavailable calcium ions,
a source of bioavailable phosphate ions, and
an antibacterial natural extract of totara.

In a further embodiment, the oral healthcare product substantially dissolves or mixes when introduced into the oral cavity such that no solid residue is left in the oral cavity

In a further embodiment, the oral healthcare product is in the form of a unit dose.

In a further embodiment, the oral healthcare product is packaged in a dispenser which dispenses unit doses.

In one embodiment, the saliva activating agent is present in a range of about 30% to about 80% w/w. In a further embodiment, the saliva activating agent is present in a range of about 40% to about 75% w/w. In a further embodiment, the saliva activating agent is present in a range of about 45% to about 70% w/w.

In a further embodiment, the saliva activating agent is non-fermentable by bacteria in the oral cavity.

In a further embodiment, the saliva activating agent is a plaque adhesion reducing agent.

In a further embodiment, the saliva activating agent is a non-fermentable carbohydrate.

In a further embodiment, the saliva activating agent is xylitol.

In a further embodiment, the oral healthcare product includes a further saliva activating agent, for example a betaine.

In a further embodiment, the pH adjusting agent is present in a range of about 1% to about 50% w/w. In a further embodiment, the pH adjusting agent is present in a range of about 5% to about 50% by weight.

In a further embodiment, the pH adjusting agent is present in a range of about 5% to about 40% by weight.

In a further embodiment, the pH adjusting agent is sodium bicarbonate.

In a further embodiment, the source of bioavailable calcium ions and the source of bioavailable phosphate ions is provided in the same agent.

In a further embodiment, the source of bioavailable calcium ions and the source of bioavailable phosphate ions is calcium glycerophosphate.

In a further embodiment, the source of bioavailable calcium ions and/or the source of bioavailable phosphate ions is present in a range of about 0.1% to about 30% w/w. In a further embodiment, the source of bioavailable calcium ions and/or the source of bioavailable phosphate ions is present in a range of about 1% to about 20% w/w. In a further embodiment, the source of bioavailable calcium ions and/or the source of bioavailable phosphate ions is present in a range of about 5% to about 20% w/w. In a further embodiment, the source of bioavailable calcium ions and/or the source of bioavailable phosphate ions is present in at about 10% w/w. In a further embodiment, the source of bioavailable calcium ions and/or the source of bioavailable phosphate ions is present in at about 5% w/w.

In a further embodiment, the antibacterial natural extract of totara is present in at about 0.001% to about 2%. In a further embodiment, the antibacterial natural extract of totara is present in at about 0.05% to about 1%. In a further embodiment, the antibacterial natural extract of totara is present at about 0.5%. In a further embodiment, the antibacterial natural extract of totara is present at about 0.1%.

In a further embodiment, the unit dose of the oral healthcare product provides:
about 150 mg to about 1200 mg xylitol,
about 5 mg to about 750 mg sodium bicarbonate,
about 0.5 mg to about 450 mg calcium glycerophosphate, and
about 0.005 mg to about 30 mg extract of totara.

In a further embodiment, the unit dose of the oral healthcare product provides:
about 225 mg to about 1050 mg xylitol,
about 25 mg to about 600 mg sodium bicarbonate,
about 25 mg to about 300 mg calcium glycerophosphate, and
about 0.25 mg to about 15 mg extract of totara.

In a further embodiment, the oral healthcare product includes an additional antibacterial natural extract selected from an extract of any one or more of the following: Neem tree, Commiphora myrrha, carboxy chitosan, licorice, green tea, Theobromine, Usnic acid, citrus bioflavonoid(s), Honokiol, Quercetin, Horopito, Coleus Forskolii oil, Phytospingosine, Carboxymethyl chitosan.

In a further embodiment, the additional antibacterial natural extract is an extract of licorice and/or theobromine.

In a further embodiment, the oral healthcare product is in solid form.

In a further embodiment, the oral healthcare product is in the form of a powder.

In a further embodiment, the oral healthcare product is in the form of a capsule.

In a further embodiment, the oral healthcare product is in the form of a tablet.

In a further embodiment, the oral healthcare product further includes a solvent and is in the form of a liquid.

Where a solvent is added to the formulation to dissolve the ingredients, it is in addition to the saliva activating agent, pH adjusting agent, bioavailable calcium ions, bioavailable phosphate ions, and antibacterial natural extract of totara and therefore the amount of solvent is not to be considered in the percentages of the other components, for example, the about 30% to about 98% w/w of a saliva activating agent is the quantity of saliva activating agent prior to addition of solvent.

In a further embodiment, the solvent is non-fermentable by the bacteria in the oral cavity.

In a further embodiment, the solvent is selected from any one or more of the following: polyglycerol esters, water, glycerine.

In a further embodiment, the oral healthcare product is in the form of a liquid filled capsule.

In a further embodiment, the oral healthcare product is in the form of a spray.

In a further embodiment, the oral healthcare product has a pH of about 7 to about 9. In a further embodiment, the oral healthcare product has a pH of about 7.8 to about 8.2. In a further embodiment, the oral healthcare product has a pH of about 8.

In a further embodiment, the oral healthcare product further includes a flavoring agent.

In a further embodiment, the oral healthcare product further includes any one or more of the following: a stabilising agent, a preservative, a filler.

According to a second broad aspect of the invention, there is provided an oral healthcare method comprising the step of:
introducing the product of the first broad aspect into the oral cavity at least once in a 24 hour period.

In one embodiment, the product is introduced into the oral cavity at least three times in a 24 hour period.

In a further embodiment, the product is introduced into the oral cavity between three and five times in a 24 hour period.

In a further embodiment, the product is introduced into the oral cavity in the form of unit doses. In a further embodiment, the product is introduced into the oral cavity in the form of one or two unit doses.

In a further embodiment, the product dissolves in the oral cavity in less than 10 minutes. In a further embodiment, the product dissolves in the oral cavity in between about 10 minutes and about 10 seconds. In a further embodiment, the product dissolves in the oral cavity in between about 7 minutes and about 10 seconds. In a further embodiment, the product dissolves in the oral cavity in less than about 3 minutes and of being introduced into the oral cavity. In a further embodiment, the product dissolves in the oral cavity in about 3 minutes to about 10 seconds of being introduced into the oral cavity.

In a further embodiment, the product is introduced into the oral cavity within about 30 minutes of eating or drinking.

In a further embodiment, the product is introduced into the oral cavity within about 10 minutes of eating or drinking.

In a further embodiment, the product is not expectorated from the oral cavity.

In a further embodiment, the method provides between about 3 to about 5 grams of xylitol per day.

According to a third broad aspect of the invention, there is provided a method of producing an oral healthcare product, comprising:
combining the following components: about 30% to about 98% w/w of a saliva activating agent, a pH adjusting agent, a source of bioavailable calcium ions, a source of bioavailable phosphate ions, and an antibacterial natural extract of totara.

In a further embodiment, the method further includes forming the components into a unit dose.

In a further embodiment, the method further includes putting the components in a dispenser which dispenses a unit dose.

In one embodiment, the saliva activating agent is present in a range of about 30% to about 80% w/w. In a further embodiment, the saliva activating agent is present in a range of about 40% to about 75% w/w. In a further embodiment, the saliva activating agent is present in a range of about 45% to about 70% w/w.

In a further embodiment, the saliva activating agent is xylitol.

In a further embodiment, the pH adjusting agent is present in a range of about 1% to about 50% w/w. In a further embodiment, the pH adjusting agent is present in a range of about 5% to about 50% by weight. In a further embodiment, the pH adjusting agent is present in a range of about 5% to about 40% by weight.

In a further embodiment, the pH adjusting agent is sodium bicarbonate.

In a further embodiment, the source of bioavailable calcium ions and the source of bioavailable phosphate ions is provided in the same agent.

In a further embodiment, the source of bioavailable calcium ions and the source of bioavailable phosphate ions is calcium glycerophosphate.

In a further embodiment, the source of bioavailable calcium ions and/or the source of bioavailable phosphate ions is present in a range of about 0.1% to about 30% w/w. In a further embodiment, the source of bioavailable calcium ions and/or the source of bioavailable phosphate ions is present in a range of about 1% to about 20% w/w. In a further embodiment, the source of bioavailable calcium ions and/or the source of bioavailable phosphate ions is present in a range of about 5% to about 20% w/w. In a further embodiment, the source of bioavailable calcium ions and/or the source of bioavailable phosphate ions is present in at about 10% w/w. In a further embodiment, the source of bioavailable calcium ions and/or the source of bioavailable phosphate ions is present in at about 5% w/w.

In a further embodiment, the antibacterial natural extract of totara is present in at about 0.001% to about 2%. In a further embodiment, the antibacterial natural extract of totara is present in at about 0.05% to about 1%. In a further embodiment, the antibacterial natural extract of totara is present at about 0.5%. In a further embodiment, the antibacterial natural extract of totara is present at about 0.1%.

In a further embodiment, the method includes additional of a further antibacterial natural extract selected from an extract of any one or more of the following: Neem tree, Commiphora myrrha, Carboxy-chitosan, licorice, green tea, Theobromine, Usnic acid, citrus bioflavonoid(s), Honokiol, Quercetin, Horopito, Coleus Forskolii oil, Phytospingosine, Carboxymethyl chitosan.

In a further embodiment, the components are in the form of a powder.

In a further embodiment, the method further includes putting the components into a capsule.

In a further embodiment, the method further includes forming the components into a tablet.

In a further embodiment, the method further includes adding a solvent such that the components are in the form of a liquid.

Where a solvent is added to the formulation, it is in addition to the saliva activating agent, pH adjusting agent, bioavailable calcium ions, bioavailable phosphate ions, and antibacterial natural extract of totara and therefore the amount solvent is not to be considered in the percentages of the other components, for example, the about 30% to about 98% w/w of a saliva activating agent is the quantity of saliva activating agent prior to addition of solvent.

In a further embodiment, the solvent is selected from any one or more of the following: polyglycerol esters, water, glycerine.

In a further embodiment, the method further includes putting the liquid into a capsule.

In a further embodiment, the method further includes putting the liquid into a dispenser.

As used herein the term “and/or” means “and” or “or”, or both.

As used herein “(s)” following a noun means the plural and/or singular forms of the noun.

The term “comprising” as used in this specification means “consisting at least in part of”. When interpreting statements in this specification which include that term, the features, prefaced by that term in each statement, all need to be present, but other features can also be present. Related terms such as “comprise” and “comprised” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

To those skilled in the art to which the invention relates, many changes in construction and widely differing embodiments and application of the invention will suggest themselves without departing from the scope of the invention as defined in the appended claims. The disclosures and the descriptions herein are purely illustrative and are not intended to be in any sense limiting.

Other aspects of the invention may become apparent from the following description which is given by way of example only and with reference to the accompanying figures and examples.

### BRIEF DESCRIPTION OF THE FIGURES

Embodiments of the invention will now be described, by way of example only, with reference to the accompanying drawings in which:

FIG. 1 shows activity of the tablet of the invention and serum of the invention on 2 hour biofilm compared to chlorhexidine digluconate, a commercially available mouthwash and a commercially available toothpaste.

FIG. 2 shows activity of the tablet of the invention and serum of the invention on 6 hour biofilm compared to chlorhexidine digluconate, a commercially available mouthwash and a commercially available toothpaste.

### DETAILED DESCRIPTION OF PREFERRED EMBODIMENTS

The oral healthcare product and method of the invention provide a safe and convenient means to maintain or re-establish healthy parameters in the oral cavity. The oral healthcare product uses about 30% to about 98% w/w of a saliva activating agent together with a pH adjusting agent, a source of bioavailable calcium ions, a source of bioavailable phosphate ions, and an antibacterial natural extract of totara to achieve this result.

The oral cavity is host to a large and diverse population of bacteria which form bio-film on any available surfaces, for example, teeth, gums, tongue, and/or cheek(s). Reference to the “oral cavity” throughout the specification should be taken to include teeth, gums, tongue, and/or cheek(s). The biofilm can contain both beneficial and detrimental bacteria. The beneficial bacteria both live in and help to create a beneficial or at least non-detrimental environment in the oral cavity, for example, a neutral pH, high mineral levels. Beneficial bacteria are mostly gram positive bacteria, are generally aerobic, are non-acidogenic, and do not cause an inflammatory response in the human body. Examples of beneficial bacteria include (but are not limited to) Streptococcus Gordonii, Strep. Oralis, Strep Mitis, Veillonella Parvula, Actinomyces Odontolyticus, Fusobacterium Nucleatum. Detrimental bacteria both thrive in and help to create a detrimental environment, for example low pH, low mineral levels. Detrimental bacteria are generally gram negative. Many detrimental bacteria are acidogenic (acid producing), aciduric (acid-tolerating), for example (but not limited to) mutans streptococci (such as Strep. mutans and Strep. sobrinus) and Lactobacilli (such as Lactobacillus Rhamnosus). Many are anaerobic (grow in absence or low levels of oxygen) for example (but not limited to) Porphyromonas Gingivalis (the main periodontal disease pathogen), Bacteroides Forsythus, Actinobacillus ActinoMycetemComitans. Some detrimental bacteria are facultative anerobes (can grow in presence or absence of Oxygen) like Mutans Streptococci. The detrimental bacteria are a causative factor in tooth decay, gum disease, bad breath and demineralisation. If attempts are made to eradicate the bacteria in the oral cavity, bacteria (and other pathogens) re-infect the oral cavity within a matter of hours. Some oral health products seek to clear the oral cavity of all bacteria in a non-selective fashion. The inventors believe this is actually detrimental to the health of the oral cavity, since it is often the detrimental bacteria which have a faster re-infection rate and a faster growth rate than the beneficial bacteria. The invention seeks preferably not to kill all bacteria within the oral cavity, but preferably to favour and promote the colonisation of the beneficial over the detrimental bacteria, hence acting as a natural prebiotic.

When there is a shift in the conditions within the oral cavity which could promote the growth of detrimental bacteria (and other detrimental pathogens) the invention seeks to quickly and efficiently re-establish the balance of conditions to slow the growth of the detrimental bacteria and promote the growth of the beneficial bacteria. Conditions which will promote the growth of detrimental bacteria can be caused by environmental factors, for example consumption of acidic foods, or can be caused by health conditions, for example disease, dry mouth and genetic conditions.

Saliva acts as a natural defence in the oral cavity, by breaking down food, buffering acids from diet and bacteria to maintain a healthy neutral or near neutral pH in the oral cavity (i.e. about pH 6.8-7.2), providing bioavailable minerals (dissolved in the saliva) to remineralise teeth and providing oxygen and antibodies that help fight detrimental bacteria in the oral cavity and around the gum-line. It is also a lubricant in the oral cavity. While saliva is naturally occurring and production is usually stimulated in response to eating (stimulated saliva), environmental issues, for example alcohol, caffeine, smoking and diet, and natural or induced physiological variation, affect the levels of saliva produced and the quality of that saliva (i.e. the ability to perform the beneficial functions discussed above).

It has therefore been found to be beneficial to activate/stimulate the production of saliva in the oral cavity and in addition enhance/encourage the saliva's ability to provide the beneficial functions discussed above. The product and method of the invention seek to reduce the erosive effects of dietary and other acids on teeth, by repeatedly adjusting the pH of the oral cavity and saliva to neutral after such attacks. Further the invention seeks to stimulate and enhance natural saliva to combat the effect of dry mouth. While these problems can be minimised by lifestyle choices, it is unlikely they can be prevented, there is therefore a need for a product and/or a method which can re-establish the balance of beneficial conditions within the oral cavity in a fast and convenient fashion.

While not wishing to be bound by theory, it is believed the methods and products of the invention act by increasing the production of saliva in the oral cavity. Further the saliva either produced as a result of the method/product or occurring naturally is enhanced by increasing the buffering capacity of the saliva, and/or increasing the mineral content in the saliva and/or increasing the plaque-adhesion reducing ability of the saliva. Such saliva is referred to herein as “enhanced saliva”. By contrast, some oral health products are desiccating (i.e. they lower the levels of saliva in the oral cavity), and as a result the beneficial effects of encouraging and activating saliva (and/or enhanced saliva) do not occur in these products.

Stimulated saliva (produced naturally when eating) is alkaline (pH 7.8-8.5) and contains bicarbonate, calcium and phosphate ions to buffer acids from the diet and bacteria that break down the food. These ions also help remineralise teeth after the acid attack. Ideally the oral environment is maintained in a neutral or near neutral pH (6.8-7.2) most of the time. Low pH (below about 5.5) in the oral cavity will cause demineralisation of the teeth, as it is believed the minerals will be leached out of the teeth at such low pH. In addition, at acidic pH (lower than neutral) remineralisation will not occur (or be limited to a low level). With excessive dietary intake of acids, partially due to changing dietary habits, and the increased use of saliva inhibiting agents (alcohol, caffeine, drugs, smoking, desiccating oral agents) many people do not get adequate protection against the attack of detrimental bacterial biofilm and dietary acids from their own naturally produced/stimulated saliva. The products and/or methods of the invention mimic and enhance the functions and ingredients of stimulated saliva.

The saliva activating agent in the product and method of the invention acts to increase or stimulate saliva to act in a defensive manner as described above. The saliva activating agent is preferably non-acidic. In a preferred embodiment the saliva activating agent is not fermentable by the bacteria in the oral cavity as fermentation by detrimental bacteria can form acids. The saliva activating agent in the product is preferably also a caries reducing agent and/or a plaque adhesion reducing agent. The saliva activating agent is preferably present in a range of about 30% to about 80% w/w, more preferably a range of about 40% to about 75% w/w most preferably a range of about 45% to about 70% w/w.

The most preferred saliva activating agent is xylitol, since xylitol is both non-fermentable by the bacteria in the oral cavity, acts as a caries reducing agent and has plaque adhesion reducing properties. The cooling effect and sweet taste of xylitol activate the production of saliva in the oral cavity. In addition, xylitol acts as a remineralisation enhancing agent. It has been shown that calcium and phosphate ions bind to the teeth more quickly and efficiently when xylitol is present.

Reference to “non-fermentable” should be taken to include low levels of fermentation and should be taken to mean a substance that is not broken down (or only broken down to a small extent) by bacteria to produce an acidic by-product. Glucose and fructose are examples of highly fermentable carbohydrates. It is preferred that the saliva activating agent is a non-fermentable carbohydrate. Xylitol is an example of a non-fermentable carbohydrate.

In a preferred embodiment, the product and the methods of the invention also contains a further saliva activating agent to stimulate saliva in the oral cavity. This is particularly preferred in the tablet or capsule form which is likely consumed without additional water. A preferred additional saliva activating agent is betaine. Betaines are particularly preferred as in some instances they have been shown to have antibacterial properties.

The amount of the pH adjusting agent can be selected to provide the required pH when the product is introduced into the oral cavity and to providing a buffering action to maintain (at least temporarily) the oral cavity at the required pH. The pH adjusting agent in the product of the invention adjusts the pH in the oral cavity and buffers the pH to maximise the beneficial effect of the bioavailable calcium ions and bioavailable phosphate ions. This is particularly important where the oral cavity is very acidic, for example after consuming acidic food or drink. In addition, an acidic environment in the oral cavity is known to promote the growth of harmful/detrimental bacteria in the biofilm. The pH adjusting agent in the product and method of the invention is present in a range of about 1% to about 50% by weight, more preferably a range of about 5% to about 50% by weight, most preferably a range of about 5% to about 40% by weight. In a preferred embodiment the pH adjusting agent is sodium bicarbonate. Sodium bicarbonate is particularly preferred as a pH adjusting agent/buffering agent because it is a food grade substance, which is considered safe for human consumption (at the levels used in the invention). It is also naturally occurring in stimulated saliva.

The source of bioavailable calcium ions and bioavailable phosphate ions is selected to provide mineral ions which are dissolved in the saliva in the oral cavity (i.e. are bioavailable). Calcium and phosphate compounds which do not dissolve in the saliva in the oral cavity will not be bioavailable. The source of bioavailable calcium ions and the source of bioavailable phosphate ions provide a high level of ions in the saliva to reduce or mitigate demineralisation in the oral cavity. The source of bioavailable calcium ions and/or the source of bioavailable phosphate ions is present in the product and methods of the invention in a range of about 0.1% to about 30% by weight, preferably about 1% to about 20% by weight, more preferably about 5% to about 20% by weight, more preferably about 10% by weight or about 5% by weight. As discussed above stimulated saliva naturally includes both calcium and phosphate ions. The product of the invention therefore replaces calcium and phosphate ions in stimulated saliva where there is a deficiency, or enhances the quantity of calcium and phosphate ions in natural stimulated saliva. The dissolved ions are readily available for absorption in the oral cavity, preferably by the teeth, if there is a deficiency in the mineral levels. The source of bioavailable calcium ions and/or the source of bioavailable phosphate ions can therefore also act as a remineralising agent depending on the level of minerals in the teeth. If there is are sub-optimal levels of minerals in the teeth and there is a higher level in the oral cavity due to the use of the products of the invention, the source of bioavailable calcium ions and/or the source of bioavailable phosphate ions will act as a remineralising agent. As discussed above, low pH (below about 5.5) in the oral cavity will cause demineralisation of the teeth, as it is believed the minerals will be leached out of the teeth at such low pH. In addition, at acidic pH (lower than neutral) remineralisation will not occur (or be limited to a low level). The pH of the oral cavity (which is affected/regulated by the product and method of the invention) will therefore affect the availability of the mineral ions. The pH of the saliva (which is controlled by the pH adjusting agent) is therefore important to the effectiveness of the source of bioavailable calcium ions and/or the source of bioavailable phosphate ions. Calcium glycerophosphate is a particularly preferred source of both bioavailable calcium ions and bioavailable phosphate ions. Calcium glycerophosphate is particularly preferred as it is very soluble in saliva (so is readily bioavailable), it provides both calcium and phosphate ions in a single compound and because it is considered a safe food grade ingredient.

Many oral health products contain components to provide fluoride ions as these are believed to be beneficial to dental health. However, the product and method of the invention preferably does not contain or produce fluoride ions. Fluoride ions are toxic in higher levels, which can be a particular problem in children. The products of the invention are intended to be swallowed without detriment to health. Detriment to health would be a probable outcome were fluoride to be included. The products of the invention are intended to be consumed at least on a daily basis and preferably twice a day or more preferably three times a day.

The antibacterial natural extract of totara used in the invention provides safe, non-toxic antibacterial activity which compliments the action of the saliva activating agent, pH adjusting agent, bioavailable calcium ions, and bioavailable phosphate ions to maintain or adjust to a healthy environment in the oral cavity. The active agent in extract of totara is believed to be (4bS,8aS)-4b,8,8-Trimethyl-1-propan-2-yl-5,6,7,8a,9,10-hexahydrophenanthren-2-ol (also known as Totara-8,11,13-trien-13-ol, sold under the trade name Totarol [available from Mende Biotech Ltd of Carterton, New Zealand, www.totarol.com], which may be extracted from the Podocarpus totara tree, other species of Podocarpaceae and Cupressaceae). The antibacterial natural extract of totara is preferably present in the product and methods of the invention at about 2% to about 0.001%, preferably about 2% to about 0.05%, more preferably at about 1% to about 0.05%, most preferably at about 0.5% to about 0.05%, preferably about 0.5% or about 0.1%. It is a particular advantage that the extract of totara can be used at low levels in the product and method of the invention in the oral cavity. This advantage is due to the ability of the extract to combine with the other components of the composition and provide an enhanced oral health environment together with the ability to ingest the product with no detrimental effect to the consumer. It is an additional advantage that the extract of totara even when provided at low levels (about 0.5% to about 0.05%) has a preservative effect on the product itself potentially allowing for an improved shelf life without the use of additional preservatives or like materials. The impact of the totarol is particularly shown in the antibacterial and antifungal efficacy shown in Example 7. As can be seen in the this Example levels of about 0.5% to about 0.05% (0.1% in Example 7) can be used in the solid product (tablet form) and levels below about 2% (not including solvent) can be used in a liquid product. Such low levels of an antibacterial product are beneficial to the user as this limits any detrimental effect on ingestion. It is particularly surprising that levels of below 0.5% of the natural extract of totara in the solid form of the product provide the efficacy shown.

While reference is made to “antibacterial natural extracts of totara” this should also be taken to include substances which have been synthetically produced to mimic the naturally extract. For example, the active compound (4bS,8aS)-4b,8,8-Trimethyl-1-propan-2-yl-5,6,7,8a,9,10-hexahydrophenanthren-2-ol, can be extracted from certain trees as discussed above, but the invention should also be taken to include the active compound when produced synthetically.

The oral healthcare product of the invention preferably kills or inhibits detrimental bacteria in the oral cavity, for example Strepococcus mutans. However, the product preferably has less or no activity against the beneficial bacteria in the oral cavity (i.e. bacteria which do not produce acids).

Additional antibacterial natural extracts which can optionally be used in the invention include, but are not limited to, Neem extract, Commiphora myrrha, carboxy chitosan, licorice, green tea (for example, epigallocatechin gallate), Theobromine, Usnic acid, citrus bioflavonoid(s), Honokiol, Quercetin, Horopito extract, Coleus Forskolii oil, Phytospingosine, Carboxymethyl chitosan. The additional antibacterial natural extract is preferably present in the invention at about 2% to about 0.001%, more preferably at about 1% to about 0.05%, most preferably at about 0.5% or about 0.1%. However, it will be apparent that the levels of antibacterial natural extract present in the invention will be dependent on the nature and activity of antibacterial natural extract used. When provided with the information in the present application, it would be within the capability of a person skilled in the art to determine the level of antibacterial natural extract to be used. As previously noted, while reference is made to “natural extracts” this should also be taken to include substances which have been synthetically produced to mimic a naturally occurring substance.

In preferred embodiments the product will also have antifungal activity. Fungi, for example but not limited to oral candidiasis, can also cause damage to the oral cavity and upset the conditions which will promote the growth of beneficial bacteria. The antifungal activity is provides by at least one, or preferably more than one of the ingredients in the product. Preferably the antifungal activity is provided by one or more of xylitol, sodium bicarbonate, and the natural extract of totara.

The product of the invention is preferably in the form of a tablet, capsule, powder, liquid filled capsule or liquid. Reference to “liquid” should be taken to include gels and serums. In the most preferred embodiments the product in is the form of a single or unit dose which can be conveniently carried by the consumer during the course of their daily activities, for example a tablet, capsule, liquid filed capsule, liquid filled tube. Alternatively the oral healthcare product is packaged in a dispenser which dispenses a unit dose. This allows for the consumer to readily carry and access the product so that it can be taken within a short time of any activity which is likely to shift the conditions in the oral cavity to those which would promote the growth of detrimental bacteria (for example eating, drinking, smoking). In such individual (unit dose) form dosing is easy and does not require additional measures or containers.

The product is preferably held in the oral cavity where it dissolves or mixes with naturally produced saliva (or saliva which is activated by the product). The product is preferably not spat out and/or rinsed away, in order to allow the active ingredients to have maximum benefit. The product is particularly convenient, as it is not necessary (or advisable) to rinse out or spit out the product, therefore wash facilities or waste disposal facilities are not required. Preferably all of the ingredients in the product dissolve or mix in the oral cavity so that there is no residue which must be expectorated (for example a gum formulation is expectorated). Further all of the ingredients are non-toxic and/or food grade, which is safe to consume in moderate quantities. The product preferably does not require the consumer to chew on the product (though the consumer may choose to bite into the product) as prolonged chewing can cause damage to the jaw joints and attrition of the teeth.

The tablet or capsule form of the product is particularly convenient for consumers which are not able to readily access more traditional (and less convenient) forms of oral care, for example tooth paste. Such consumers would include workers on long shifts, hikers, and soldiers while on active service. The product of the invention allows for the convenient and easy care of the oral cavity with little effort on the part of the consumer. It allows for high compliance of the method of the invention by the consumer. While it is not intended to replace the brushing and flossing of teeth, which clears debris from the oral cavity, the product of the invention is more conveniently used at multiple times during a day than traditional forms of oral healthcare. The products of the invention can also be used as a replacement for traditional toothpaste. The product can be introduced into the oral cavity and a toothbrush can be used to spread the product around the oral cavity. This is particularly convenient when away from the home environment where it is not convenient to carry multiple products.

Where the product of the invention is in solid form, (for example, powder or tablet) the product dissolves when introduced into the oral cavity, such that no solid residue is left in the oral cavity. It is particularly desirable for the product to dissolve in the oral cavity in order to reduce or prevent abrasion of the protective outer coating (enamel) of the teeth. In such a preferred form the product does not contain abrasive components, for example silica. In addition, it is preferable that the product dissolve in the oral cavity to allow maximal bioavailability of the minerals in the remineralising agent. Maximal bioavailability will allow of more effective remineralisation of the teeth.

In the most preferred form the invention will be in substantially dry solid form (for example powder or tablet). This allows for ease of storage and transport and an extended shelf life either without the need for additional preservatives or with less quantity of preservative.

Where the product is in tablet form the product of the invention optionally contains one or more tableting aids. Tableting aids include, but are not limited to, binders, (for example silicified microcrystalline cellulose, microcellulose), disintegrants (for example corn starch, carboxymethylcellulose, calcium carboxymethylcellulose, sodium carboxymethyl starch, croscarmellose sodium {e.g., Ac-Di-Sol}, crospovidone, low-substituted hydroxypropylcellulose (L-HPC), hydroxypropylstarch and the like), lubricants (for example magnesium stearate, calcium stearate, talc, sucrose esters of fatty acids, sodium stearyl fumarate and the like). However, these are generally to be kept to a minimum and are optionally included for the purposes of providing the product in a convenient form.

The products of the invention in the tablet can be produced by appropriately combining operations such as granulation (wet or dry), mixing, capsule filling, compression molding, direct compression, coating and the like. For granulation, for example, a granulation machine such as a high-shear granulator, a fluid bed granulator and the like is used.

In preferred embodiments where the product is a tablet or capsule, the product is optionally coated. For example, where the product is a tablet a film coating apparatus is optionally used. A film coating may be applied using a film coating apparatus. Examples of a coating base include: a polyol coating base, aqueous film coating base, enteric film coating base, sustained-release film coating base and similar. An example of an aqueous film coating base include cellulose polymers such as hydroxypropylcellulose. Use of a film coating is particularly preferred where the product is a tablet as the coating can minimize the uptake of moisture when the product is hydroscopic is nature.

Where the product is in liquid form the product will include an additional solvent which dissolves the components, is non-toxic, is preferably non-desiccating and is preferably non-fermentable by the bacteria in the oral cavity. Examples of suitable solvents include (but are not limited to polyglycerol esters, water and glycerine (preferably vegetable glycerine). Where the product is in liquid form, it is preferably miscible with the saliva in the oral cavity, such that no solid residue is left in the oral cavity, and to provide readily bioavailable mineral ions, and have a pleasant feel in the mouth. The liquid form is optionally provided in a multiple use container (for example a container which dispenses a unit dose, such as a pump or spray) or in single use capsules or small containers (for example tubes and sachets). The liquid form of the product is particularly beneficial for consumers with a more delicate oral cavity, for example, infants and/or the elderly. Some consumers will simply prefer the mouth feel of a liquid product, for example in the form of a serum. Where a solvent is added to the formulation to dissolve the components, it is in addition to the saliva activating agent, pH adjusting agent, bioavailable calcium ions, bioavailable phosphate ions, and antibacterial natural extract of totara and therefore the amount of solvent is not to be considered in the percentages of the other components, for example, the about 30% to about 98% w/w of a saliva activating agent is the quantity of saliva activating agent prior to addition of solvent.

Examples of flavoring agents that are optionally used in the invention include Anethole, Peppermint, Spearmint, Thyme, Licorice, sage, Tulsi, Heohesperidin Dihydrochalcone. Preferably any flavoring agent(s) used will not be substantially fermentable by the bacteria in the oral cavity. When flavorings, for example natural sugars, are fermented by bacteria in the oral cavity, acids are produced which cause the pH in the oral cavity to lower. The use of flavoring agents, while not directly contributing to the oral health benefits of the invention, help with compliance with the method by making the product more pleasant to take. This is particularly the case for children.

The products can optionally also contain further components, for example but not limited to, the product can optionally contain one or more a stabilising agent, a preservative and/or a filler. The product can optionally contain further beneficial agents, for example, but not limited to, zinc citrate and/or de-sensitizing agents, for example potassium salts.

The invention provides an oral healthcare method which includes introducing the product of the invention into the oral cavity at least once in a 24 hour period. However, in a more preferred embodiment the product is introduced into the oral cavity at least three times in a 24 hour period, more preferably between three and five times in a 24 hour period. The convenience of the product, particularly when in tablet and capsule form, allows for the product to be easily transported so that the consumer can take it with them during their daily activities. In a most preferred for of the invention, the product is introduced into the oral cavity within about 30 minutes of any activity which will either dry out the oral cavity or lower the pH of the oral cavity (for example eating, drinking or smoking), more preferably within about 10 minutes of any of these activities. When the product is in tablet, capsule (or any dose measured form/unit dose), at each occurrence of taking the product it is optionally in one or two doses. For example, where the product in tablet is consumed three times a day after meals, at each occasion either 1 or 2 tablets is taken. In this example a minimum of 3 tablets is consumed and a maximum of 6 tablets is consumed. This will be at the preference of the consumer. In the preferred embodiment the method of the invention provides a total of about 3 to about 5 grams of xylitol to the consumer per 24 hours. Preferably the about 3 to about 5 grams of xylitol per day is consumed in portions (i.e. doses, for example a tablet) over the course of 24 hours, rather than in a single dose.

The product preferably dissolves in the oral cavity in less than about 10 minutes, preferably between about 10 minutes and about 10 seconds, preferably between about 7 minutes and about 10 seconds, preferably less than about 3 minutes, preferably within about 3 minutes to about 10 seconds of being introduced into the oral cavity. However, it will be apparent that the dissolve time will be dependent on how the product is consumed. It may dissolve slower if the consumer does not move or crush the product once it has been placed in the oral cavity, it may dissolve faster if the consumer imminently crushes the product, for example it may dissolve in about 3 minutes to about 2 seconds.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavor in any country in the world.

The invention may also be said broadly to consist in the parts, elements and features referred to or indicated in the specification of the application, individually or collectively, in any or all combinations of two or more of said parts, elements or features.

Wherein the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the scope of the invention.

Most preferred combinations comprise (but are not limited to):

(a) an oral healthcare product in solid form, comprising:
about 30% to about 80% w/w of a saliva activating agent (e.g. xylitol),
about 1% to about 50% pH adjusting agent (e.g. sodium bicarbonate),
about 1% to about 20% of a source of bioavailable calcium ions and phosphate ions (e.g. calcium glycerophosphate),
about 0.05% to about 0.5% antibacterial natural extract of totara;

(b) an oral healthcare product in liquid form, comprising:
about 30% to about 80% w/w (prior to addition of solvent) of a saliva activating agent (e.g. xylitol),
about 5% to about 50% (prior to addition of solvent) pH adjusting agent (e.g. sodium bicarbonate),
about 1% to about 20% (prior to addition of solvent) of a source of bioavailable calcium ions and phosphate ions (e.g. calcium glycerophosphate),
about 0.05% to about 2% (prior to addition of solvent) antibacterial natural extract of totara; solvent.

### EXAMPLES

### Example 1

### Powder Form

Table 1 shows an example of a formulation for a product of the invention in powder form. The menthol and menthol flavor are optionally included. The menthol and menthol flavor can alternatively be replaced with other flavoring agents. If other flavoring agents are used these are preferably non-fermentable by the bacterial in the oral cavity.

The powder form is preferably packaged into unit dose form for convenience and portability, which promotes compliance with the preferred oral healthcare method of taking the product multiple times during the day, most preferably within 30 minutes of actives which upset the natural balance in the oral cavity (for example eating, drinking and smoking). However, even where the powder form is not packaged in unit dose form, it will dissolve in the oral cavity, such that it is not necessary (or advisable) to rinse out or spit out the product. Thus, even where the product is not in unit dose form it is still quick and convenient to take multiple times during the day.

TABLE 1

| | Amount % w/w | Amount % w/w |
|---|---|---|
| | (Flavored Powder | (Flavored Powder |
| Ingredient | Formulation 1) | Formulation 2) |
| Xylitol | 50.00 | 51.90 |
| Sodium Bicarbonate | 37.50 | 37.50 |
| Calcium Glycerophosphate | 10.00 | 10.00 |
| Menthol | 2.00 | 0.50 |
| Menthol Flavor | 0.40 | 0 |
| Totarol | 0.10 | 0.10 |

The following is an example of the manufacturing method for the product of the invention in powder form:

Ensure equipment is properly sanitized
Add the following ingredients in the following order or to suit particular operation
Add Xylitol
Add Sodium Bicarbonate
Add Calcium Glycerophosphate
   Ensure ingredients undergo thorough mixing
   While mixing activate spray—Spray Alcohol/Menthol/Totarol blend
   Ensure fine and consist spray procedures while product is mixing
   Continue to mix for 5 minutes after Alcohol dispersion completed
   Access mixture for consistency and dryness
   Fill into 75 gram containers

Minor ingredients (i.e. where small quantities) can be dispersed in ethanol or a similar safe solvent and sprayed onto the powder during the blending process to provide even dispersion, then dried thoroughly. For example totarol can be dispersed in ethanol and sprayed into mixture. The ethanol will substantially evaporate during the process so will not be present (beyond very small amounts) in the final product.

### Example 2

### Tablet Form (Unit Dose)

Table 2 shows examples of formulations for a product of the invention in unit dose tablet form.

TABLE 2

| | | | Amount % w/w |
|---|---|---|---|
| | Amount % w/w | Amount % w/w | (Flavored |
| | (Flavored Tablet | (Flavored Tablet | Tablet |
| Ingredient | Formulation 1) | Formulation 2) | Formulation 3) |
| Xylitol (direct | 68.9 | 66.7 | 56 |
| compression | | | |
| grade) | | | |
| Sodium | 5.0 | 5.0 | 4.5 |
| Bicarbonate | | | |
| Calcium | 10.0 | 10.0 | 7.0 |
| Glycerophosphate | | | |
| Betaine | 3.0 | 2.0 | 1.0 |
| Menthol | 1.0 | 1.0 | 1.0 |
| Menthol | 5. | 0.1 | 0.2 |
| Flavor | | | |
| (Natural) | | | |
| Totarol | 0.1 | 0.1 | 0.1 |
| Silicified | 7.00 | 15.1 | 30.2 |
| Microcrystalline | | | |
| Cellulose | | | |
| (tableting aid) | | | |

### Example 3

Table 3 gives examples of the range in size and weight of the product of the invention when in unit dose tablet form (for example, flavored tablet formulations 1, 2 and 3.

TABLE 3

| | | Approximate | Approximate |
|---|---|---|---|
| | Current | Minimum | Maximum |
| Tablet weight (mg) | 700 | 500 | 1500 |
| Tablet Diameter (mm) | 9 | 5 | 15 |

### Example 4

Tables 4 and 5 show further examples of formulations for products of the invention in tablet form. Tables 4 and 5 also show approximate minimum and maximum percentages for each of the ingredients. It will be apparent to a person skilled in the art that where the quantity of one ingredient is lowered in percentage terms another ingredient(s) will increase in percentage terms such that the sum is 100%.

TABLE 4

| | | Approximate Min | Approximate Max |
|---|---|---|---|
| INGREDIENTS | % w/w | range % w/w | range % w/w |
| Xylitol | 66.4 | 0.1 | 85 |
| Calcium | 10 | 1 | 30 |
| Glycerophosphate | | | |
| Sodium Bicarbonate | 5 | 1 | 30 |
| Menthol | 1.4 | 0.1 | 10 |
| Flavour | 5 | 0.1 | 20 |
| Talc | 1 | 0.1 | 5 |
| Magnesium Stearate | 1 | 0.1 | 5 |
| Silicified | 10 | 0.1 | 20 |
| microcrystalline | | | |
| cellulose | | | |
| (tableting aid) | | | |
| Totarol | 0.1 | 0.001 | 5 |
| Citrus Bioflavonoids | 0.1 | 0.001 | 5 |
| Total | 100 | | |

TABLE 5

| | | | Max range % |
|---|---|---|---|
| INGREDIENTS | % w/w | Min range % w/w | w/w |
| Xylitol | 66.4 | 0.1 | 85 |
| Calcium Lactate Gluconate | 7 | 1 | 30 |
| Sodium Bicarbonate | 5 | 1 | 30 |
| Menthol | 1.4 | 0.1 | 10 |
| Flavour components | 5 | 0.1 | 20 |
| Betaine | 3 | 0.1 | 20 |
| Talc | 1 | 0.1 | 5 |
| Magnesium Stearate | 1 | 0.1 | 5 |
| dibasic calcium phosphate | 10 | 0.1 | 20 |
| Totarol | 0.1 | 0.001 | 5 |
| Horopito Extract | 0-0.1 | 0 | 5 |
| Quercetin | 0-0.1 | 0 | 5 |
| Total | 100 | | |

### Example 5

### Manufacturing Method (Tablet Form)

The ingredients are generally dry mixed—for example, a mixer such as a V-type mixer, a ribbon blender, cone type/rotary or a tumbler mixer and the like is used.

The minor ingredients are added using micro-grinding and micro-blending as a dry mixture or hydrophilic and less water soluble ingredients maybe dispersed in ethanol or a similar safe solvent and sprayed onto the powder during the blending process to provide even dispersion. For example totarol can be dispersed in ethanol and sprayed into mixture. The ethanol will substantially evaporate during the process so will not be present (beyond very small amounts) in the final product.

Milling and sifting may be required prior to tableting. The tablets may be compression molded, for example punching using a single punch tableting machine, a rotary tableting machine and the like, for example at a pressure of generally 0.3-35 kN/cm2. The tablets can optionally be film coated.

The particle size can vary. In direct compression particle size is generally fine, and preferably has lubricity and good flow characteristics. The granules/particles used have a particle size of generally not less than 1000 μm for not more than 20%, not more than 150 μm for not more than 65% (with 16M sieves, on (remaining on sieves); not more than 20%; with 100M sieves, pass (pass through sieves): not more than 65%), preferably not less than 1000 μm for not more than 5%, not more than 150 μm for not more than 40% (with 16M sieves, on: not more than 5%; with 100 M sieves, pass: not more than 40%).

The following is an example of the manufacturing method for the product of the invention in tablet form.
Ensure equipment is properly sanitized
Mill the Menthol—Ensure that the menthol is finely milled
Add the following ingredients in order or to suit particular operation
   Add Calcium Glycerophosphate
   While mixing activate spray—Spray Ethanol/Totarol blend
   Ensure ingredients undergo thorough mixing
   Ensure fine and consist spray procedures while product is mixing
   Continue to until the alcohol dispersion completed
   Add Sodium Bicarbonate
   Add Xylitol
   Add Tablet aids—
Assess mixture for consistency and dryness
Add ground Menthol and continue to mix until a homogenous mixture is obtained
Tablet into 650-750 mg tablets

### Example 6

### Liquid Form (Serum)

Table 6 shows an example of a formulation for a product of the invention in liquid (serum) form.

| | Amount % w/w |
|---|---|
| | (Flavored serum Formulation 1) |
| | (percentage of components not |
| | including solvents shown in |
| Ingredient | brackets) |
| Aqua (solvent) | 69.75 |
| Xylitol | 15.00 (59.41) |
| Sodium Bicarbonate | 2.50 (9.90) |
| Vegetable Glycerine | 5.00 |
| Chitoglycan | 4.50 (17.82) |
| Carrageenan Gum | 1.00 (3.96) |
| Locust Bean Gum | 0.50 (1.98) |
| Calcium Glycerophosphate | 0.40 (1.58) |
| Menthol 25% (poyglycerl esters) | 0.40 (1.58) |
| Totarol 25% (K7 type soln - | 0.40 (1.58) |
| commercially available) | |
| Phytic acid (As Sodium Phytate) | 0.20 (0.79) |
| Zinc Citrate | 0.20 (0.79) |
| Licorice extract (or Theobromine) | 0.05 (0.20) |
| Flavour (Chocolate, Berry or | 0.10 (0.39) |
| Passion fruit) | |
| Potassium Salt (for example | 0 to 10 |
| Oratate, Cirtrate, Aspartate) | |

Method for Production of Serum—

Ensure equipment is properly sanitized
Add the following ingredients in order or to suit particular operation
Add Purified Water (Aqua)
Add Xylitol
Add Sodium Bicarbonate
Add Calcium Glycerophosphate
   Disperse with homogenisation until all ingredients are dispersed or dissolved
   Add remaining ingredients except the Gum contents
   Add Carrageenan and locust bean gum under homogenisation—until fully dispersed
   Continue to homogenise until a golden clear/translucent gel is formed
   Continue to mix for 10 minutes
   Let sit overnight—prior to filling containers

### Example 7

### Activity on Biofilm

A study based on the methodology in the following references was performed:

Ramalingam, K., Amaechi, B. T., Ralph, R. H. and Lee, V. A. (2012). Antimicrobial activity of nanoemulsion on cariogenic planktonic and biofilm organisms. Archives of Oral Biology 57: 15-22. Stepanovic, S., Cirkovic, I., Ranin, L. and Svabic-Vlahovic, M. (2004). Biofilm formation by Salmonella spp. and Listeria monocytogenes on plastic surface. Letters in Applied Microbiology 38: 428-432.

The three microbial strains (Streptococcus mutans, Lactobacillus casei and Candida albicans) were initially grown separately and then combined in 96 well plate cultures. After two different times of culturing (2 hours and 6 hours), non-adherent cells were removed and the samples at seven concentrations (0.4%, 4.0%, 10%, 20%, 40%, 80% and 100%) were then incubated with the film of the three organisms. After a defined period of time the solutions in the wells were discarded and the number of viable organisms quantified by a colorimetric assay and determined by measurement of OD595 nm using a VersaMax 96 well plate reader. The effects of the samples were assessed at each concentration in triplicate. There were an Inoculant, Sample Only Controls and a positive control (Chlorhexidine) also assayed in triplicate.

Samples Tested
1. Tablet of the invention—Six tablets (Formulation 3) were crushed and dissolved in 30 ml of distilled water which provided the 100% stock solution.
2. Serum of the Invention—The serum of the invention was suspended in distilled water so that it was less viscous. 15 gms was weighed to which an equivalent weight of distilled water (15 mls) was added to prepare the 30 mls of the 100% stock solution at 1 gm/ml.
3. Comparative Example 1—30 g toothpaste (standard Colgate) was suspended in 30 ml of distilled water to produce the 100% stock solution.
4. Comparative Example 2—Mouthwash (Listerine alcohol free) was used undiluted. 30 mls was measured for use as the 100% stock solution.
5. Comparative Example 3—Chlorhexidine digluconate came as a 20% stock solution in water.

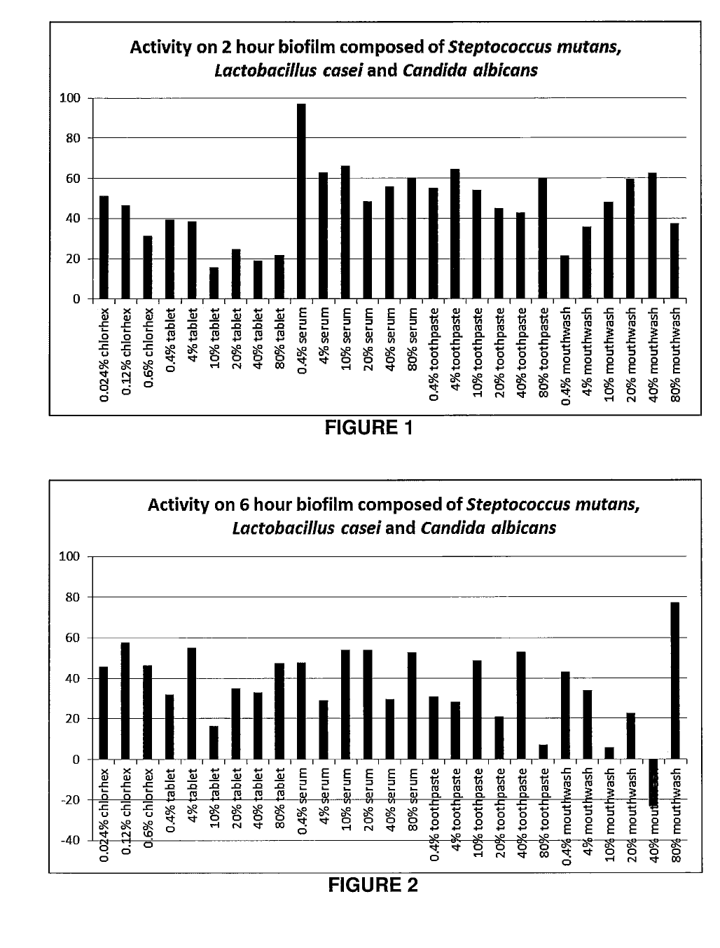

Results of the study are shown in FIGS. 1 and 2. High concentrations of each of the samples tended to produce unreliable results. Therefore the 100% concentration samples are not shown in the graphs.

The results for the tablet of the invention and the serum of the invention are shown in black. The comparative examples are shown in grey. The results show the products of the invention, including the antibacterial natural extract of totara, are comparable in effectiveness and in some cases more effective than oral health care products presently on the market. However, the products of the invention are safe to consume and are highly convenient to take at periodic intervals without the need for bathroom or waste disposal facilities. By comparison, the comparative products have instructions not to swallow and in the case of the mouthwash to seek medical help if consumed in quantities.

1-41. (canceled)

42. An oral healthcare product, comprising:
about 30% to about 98% w/w of a saliva activating agent,
a pH adjusting agent,
a source of bioavailable calcium ions,
a source of bioavailable phosphate ions,
an antibacterial natural extract of totara.

43. The oral healthcare product of claim 42 wherein the product substantially dissolves or mixes when introduced into the oral cavity such that no solid residue is left in the oral cavity.

44. The oral healthcare product of claim 42 wherein the oral healthcare product is in the form of a unit dose.

45. The oral healthcare product of claim 44 wherein the unit dose of the oral healthcare product provides:
about 150 mg to about 1200 mg xylitol,
about 5 mg to about 750 mg sodium bicarbonate,
about 0.5 mg to about 450 mg calcium glycerophosphate, and
about 0.005 mg to about 30 mg extract of totara.

46. The oral healthcare product of claim 42 wherein the saliva activating agent is present in a range of about 30% to about 80% w/w.

47. The oral healthcare product of claim 42 wherein the saliva activating agent is xylitol.

48. The oral healthcare product of claim 42 wherein the pH adjusting agent is present in a range of about 1% to about 50% w/w.

49. The oral healthcare product of claim 42 wherein the pH adjusting agent is sodium bicarbonate.

50. The oral healthcare product of claim 42 wherein the source of bioavailable calcium ions and the source of bioavailable phosphate ions is provided in the same agent.

51. The oral healthcare product of claim 42 wherein the source of bioavailable calcium ions and the source of bioavailable phosphate ions is calcium glycerophosphate.

52. The oral healthcare product of claim 42 wherein the source of bioavailable calcium ions and/or the source of bioavailable phosphate ions is present in a range of about 0.1% to about 30% w/w.

53. The oral healthcare product of claim 42 wherein the antibacterial natural extract of totara is present in at about 0.001% to about 2%.

54. The oral healthcare product of claim 42 wherein the oral healthcare product includes an additional antibacterial natural extract selected from an extract of any one or more of the following: Neem tree, Commiphora myrrha, carboxy chitosan, licorice, green tea, Theobromine, Usnic acid, citrus bioflavonoid(s), Honokiol, Quercetin, Horopito, Coleus Forskolii oil, Phytospingosine, Carboxymethyl chitosan.

55. The oral healthcare product of claim 42 wherein the oral healthcare product is in the form of a capsule or tablet.

56. An oral healthcare method comprising the step of:
introducing the oral healthcare product of claim 42 into the oral cavity at least once in a 24 hour period.

57. The oral healthcare method of claim 56 wherein the product dissolves in the oral cavity in between about 7 minutes and about 10 seconds.

58. The oral healthcare method of claim 56 wherein the product is introduced into the oral cavity within about 30 minutes of eating or drinking.

59. The oral healthcare method of claim 56 wherein the product is not expectorated from the oral cavity.

60. A method of producing an oral healthcare product, comprising:
combining the following components: about 30% to about 98% w/w of a saliva activating agent, a pH adjusting agent, a source of bioavailable calcium ions, a source of bioavailable phosphate ions, and an antibacterial natural extract of totara.

61. The method of claim 60 wherein the method further includes forming the components into a unit dose.
